(19) United States
(12) Patent Application Publication
(10) Pub. No.: US 2026/0283778 A1
(43) Pub. Date: Sep. 24, 2026

(54) SYSTEMS AND METHODS FOR MAKING A MEDICAL BALLOON, FOR EXAMPLE AN INFLATABLE VASCULAR ENDOPROSTHESIS DEVICE

(71) Applicant: Regents of the University of Minnesota, MInneapolis, MN (US)

(72) Inventors: Joseph R. Broomhead, Southport, NC (US); Rumi Faizer, Claremont, CA (US)

(73) Assignee: Regents of the University of Minnesota, Minnapolis, MN (US)

(21) Appl. No.: 19/473,557

(22) PCT Filed: Apr. 22, 2024
(86) PCT No.: PCT/US2024/025705

§ 371 Date: Oct. 8, 2025

Related U.S. Application Data
(60) Provisional application No. 63/461,096, filed on Apr. 21, 2023.

(51) Int. Cl. A61F 2/07

(52) U.S. Cl. CPC A61F 2/07; A61F 2002/077; A61F 2230/0069; A61F 2240/002; A61F 2250/0003

(57) ABSTRACT

Methods for making thin wall balloons or bladders. Methods of the present disclosure provide for the manufacture of medical balloon devices (150) from a singular elastic material, for example double walled balloons useful as vessel implants. In some non-limiting examples, the medical device balloon is formed by 3D printing. The methods of the present disclosure are customizable for individual patients and can generate medical balloon devices that can be used as venous or aortic stent-graft devices.

### BACKGROUND

The present disclosure relates to methods for making thin wall balloons or bladders. More particularly, it relates to methods for making double walled balloons or bladders appropriate for medical uses, and devices incorporating such balloons.

Customized inflatable devices are useful for a plethora of end-use applications, including as, or as part of, a medical device intended to be implanted into a patient. One format of a customized inflatable device has a hollow, generally cylindrical shape (e.g., akin to a double tube construction) defining a central passage or lumen open to opposing ends of the device. A fillable chamber or reservoir is defined between inner and outer walls of the cylinder; when a compressible fluid or other material is delivered into the chamber, a compliant volume buffer is created between the inner and outer walls. By formatting the inner wall to be flexible, the inner wall will deform radially inwards with inflation. These and similar constructions can be useful with or as a compliant vessel implant (e.g., endoluminal stent or sent graft deployed in the aorta or other blood vessel) that, following implant, mimics the compliance of a healthy vessel, for example as described in Faizer et al., U.S. Pat. No. 11,883,273, the entire teachings of which are incorporated herein by reference.

The double walled inflatable devices described above show great promise for vessel implant and other end-use applications. In theory, these and other end use applications would benefit from the provision of the inflatable device as a singular body balloon or bladder. Unfortunately, conventional manufacturing techniques are less than optimal for creating a double walled balloon, let alone a double walled balloon customized for a particular patient.

### SUMMARY

The inventors of the present disclosure have recognized that a need exists for improved methods for making double walled balloons and for devices, such as complaint vessel implants, utilizing the double walled balloons.

Some aspects of the present disclosure relate to systems, designs and methods for making a balloon that is double walled (kind of like a hydro flask water bottle as another comparison) from a singular elastic material, and balloons resulting therefrom. In some non-limiting embodiments, methods of the present disclosure utilize 3D printing, but can include or employ other manufacturing methods. The methods of the present disclosure promote customizability of the inner diameter profile, device geometry, and stiffness of the balloon. Some aspects of the present disclosure use stereolithography to create a customizable balloon that regulates pressure through its vascular profile and level of inflation, not through obstruction of the vessel wall. In some examples, the systems and methods of the present disclosure can be useful in forming an implantable, inflatable device that sits in the descending aorta of a patient with a stiff aorta as described, for example, in Faizer et al., U.S. Pat. No. 11,883,273, the entire teachings of which are incorporated herein by reference. While some embodiments described below entail a vascular device, the systems and methods of the present disclosure are useful for other applications.

In some non-limiting embodiments of the present disclosure, 3D printing or other manufacturing techniques are used to develop a novel aortic endoprosthesis from a singular elastic material that can restore compliance to a diseased aorta. For example, 3D printing technology can be employed to develop a low-profile aortic endoprosthesis that has a long enough segment of gas-filled cushion and large enough internal lumen to reduce systolic blood-pressure by 20 mmHg and reduce pulse wave velocity (PWV) by 10% in comparison to a native elderly diseased aorta. With embodiments of the present disclosure, the device design can be refined such that the device can be collapsed to a low enough profile to be constrained within an endograft delivery system.

### BRIEF DESCRIPTION OF THE DRAWINGS

FIG. 1A is a simplified perspective view of a double walled medical balloon device in accordance with principles of the present disclosure;

FIG. 1B is a simplified cross-sectional view of the balloon device of FIG. 1A, taken along the line 1B-1B;

FIG. 2 is a diagram schematically representing an example method in accordance with principles of the present disclosure for printing a medical balloon device;

FIG. 3 is a diagram schematically representing an example method in accordance with principles of the present disclosure for printing a medical balloon device;

FIG. 4 are modeled support designs in accordance with principles of the present disclosure for 3D printing a double walled medical balloon device;

FIG. 5A is a perspective view of an example double walled medical balloon device prepared in accordance with methods of the present disclosure;

FIG. 5B is a side view of the balloon device of FIG. 5A;

FIG. 5C is a top view of the balloon device of FIG. 5A;

FIG. 6A is a perspective view of an example double walled medical balloon device prepared in accordance with methods of the present disclosure;

FIG. 6B is a side view of the balloon device of FIG. 6A;

FIG. 6C is a top view of the balloon device of FIG. 6A;

FIG. 7 is a schematic view of a testing system described in the Examples section;

FIG. 8 presents graphs depicting results of the Examples section;

FIG. 9 is a graph depicting results of the Examples section;

FIG. 10 is a graph depicting results of the Examples section;

FIG. 11A is a perspective view of a double walled medical balloon device of the Examples section;

FIG. 11B is a side view of the balloon device of FIG. 11A;

FIG. 11C is a top view of the balloon device of FIG. 11A:

FIGS. 12A and 12B are graphs depicting results of the Examples section;

FIG. 13 is a graph depicting results of the Examples section; and

FIG. 14 is a graph depicting results of the Examples section.

### DETAILED DESCRIPTION

Aspects of the present disclosure provide methods for making a medical balloon device, such as a double walled balloon device, from a singular elastic material. Some non-limiting embodiments of the present disclosure utilize a novel 3D printing technique to create a medical balloon device out of an elastic material. In some non-limiting examples, a desired geometry can be achieved with a Formlabs 3BLStereolithography 3D printer to print a balloon device in an elastic resin. In other embodiments of the present disclosure, a medical balloon device, for example a double walled balloon device described below, is formed from a singular elastic material using techniques other than 3D printing, for example via molding. A wide variety of differently configured or formatted medical balloon devices can be generated with the methods of the present disclosure.

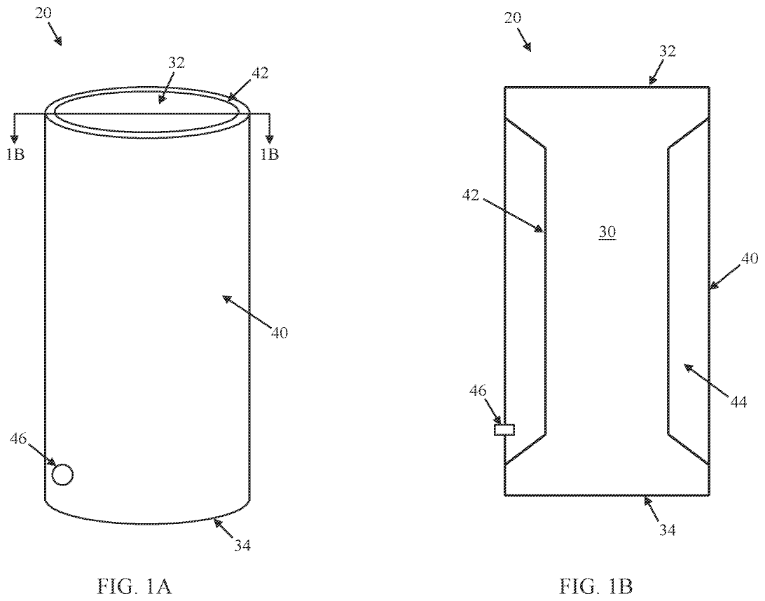

In some non-limiting examples, methods of the present disclosure can include making or manufacturing a double walled medical balloon device useful with or as a complaint vessel implant. By way of reference, one example of a double walled medical device balloon 20 of the present disclosure is shown in simplified form in FIGS. 1A and 1B. The balloon device 20 has a generally hollow cylindrical shape defining a central passage or lumen 30 that is open at opposing ends 32, 34 of the balloon device 20. The balloon device 20 includes an outer wall 40 and an inner wall 42. Opposing ends of the inner wall 42 are connected to the outer wall 40 to create a chamber or reservoir 44 between the outer and inner walls 40, 42. A port 46 is formed or provided though the outer wall 40 and is in fluid communication with the chamber 44. A rigidity of the outer wall 40 is greater than that of the inner wall 42; stated otherwise, the inner wall 42 is formed to be more compliant than the outer wall 40. With this construction, the inner wall 42 will deform radially inwardly (thus altering a diameter or shape of the central passage 30) with inflation of the chamber 44.

In some embodiments, Solidworks or similar software can be used to design each iteration of the device in 3D. In some examples, the 3D printed balloon device includes multiple walls (e.g., the outer and inner walls 30, 32) defining the chamber 44 to be filled with gas for inflation, and the port 46 at its lowest point to allow access to the chamber 46. The port 46 can be as low as possible to avoid the print from exploding within the 3D printer due to high pressure. The printed balloon device 20 can have the inner lumen walls 42 that are thin enough to allow for inflation, but thick enough to survive testing and long term use. The outer lumen wall 40 can be thick enough to remain stiff during inflation but thin enough to allow for a low profile device. In some non-limiting examples, the printed ballon device 20 can have a length on the order of 14 centimeters (cm) long to function similarly to existing stent-graft systems, and an outer diameter on the order of 26 millimeters (mm) to mimic existing stent graft systems, although any other dimensions, greater or lesser, are equally acceptable.

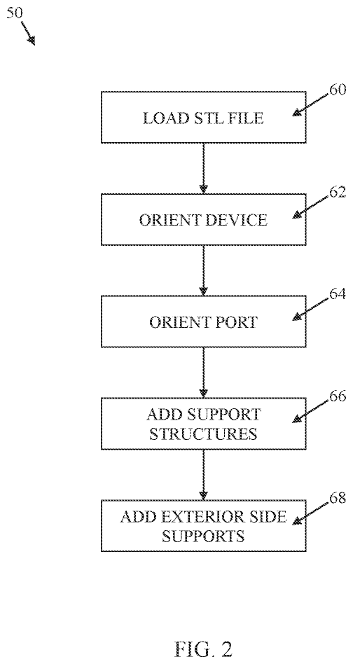

With non-limiting examples in which in which 3D printing is employed, some methods of the present disclosure for manufacturing a medical balloon (e.g., the balloon device 20) via 3D or stereolithographic (SLA) printing include setting up a print for a functional device. In some embodiments, and with reference to FIG. 2, steps for a method 50 in accordance with principles of the present disclosure for setting up the print can include one or more or all of:
1) Load the device's .stl file into the Preform software at 60. In this regard, desired dimensions or attributes can be customized.
2) At 62, the device is oriented relative to the build plate or print bed of the 3D printer. In some embodiments, the device is oriented so that the cylindrical edge is perpendicular to the build plate.
3) At 64, the device is oriented to locate the intended port (e.g., the port 46 (FIGS. 1A and 1B)) relative to the build plate. In some embodiments, the device is arranged so that the inlet port for the balloon is closest to the build plate. Due to pressure buildup due to SLA printing, the orientation of the balloon matters to prevent the device from exploding during the print.
4) At 66, customized support structures are added. In some embodiments, step 66 optionally includes ensuring that there are no internal support structures as they will form organically within the balloon. In some embodiments, step 66 can include adding support nodes to the bottom edge of the device in a circle and/or adding support structures around the inside of the device up until the farthest inward wall. With these and related examples, this will support the entire inner balloon wall.
5) At 68, custom supports are added along the sides of the device outer wall. In some examples, the device to be printed can benefit from three scaffolds from the base to the top. The custom supports can, in some embodiments, be added by manually placing support nodes along the edge.
6) The print set up methods of the present disclosure can optionally further include checking the model for printability issues. The less support the better, as the structures will need to be cut after processing which could lead to failure.
7) The print set up methods of the present disclosure can optionally further include multiplying the model for the number of devices intended to be created.

Following completion of the method 50 (or similar techniques), the print tray is autoarranged. The print is sent to the printer and the filed saved for later reference.

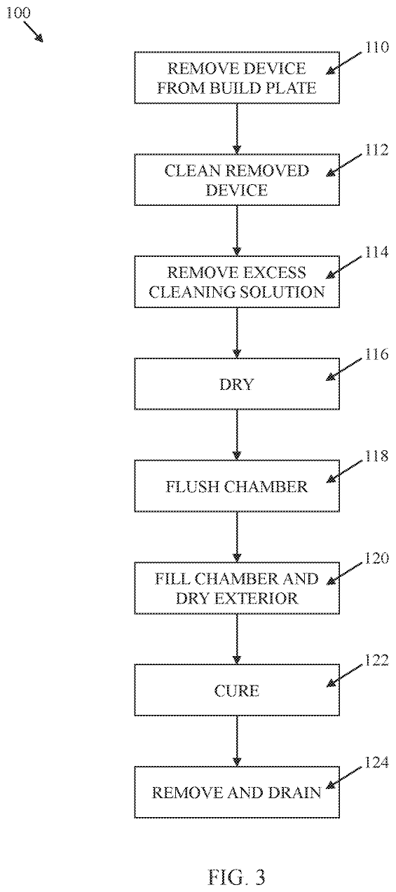

Some methods of the present disclosure can further include a post-processing procedure to achieve consistent, functional 3D printed devices. In some embodiments, and with reference to FIG. 3, a post-processing method 100 in accordance with principles of the present disclosure can include one or more or all of the following steps:
1) At 110, the printed device is removed from the build plate (e.g., Formlabs 3D Printing build plate). In some examples, a scraper can be used to carefully remove the devices from the Formlabs 3D Printing build plate, taking care to not to remove any support structures during this step.
2) At 112, the removed device is cleaned. For example, the removed device(s) is placed into a wash station (e.g., the Form Wash station), which should be filled with 99% isopropyl alcohol (IPA) or other appropriate cleaning solution formulated to remove uncured resin from the surface of the part(s). With these and related embodiments, the cleaning at step 112 can use convection to penetrate the inside of the balloon with IPA, removing the excess resin inside.
3) The cleaning step 112 can be performed for a predetermined length of time (e.g., 10 minutes).
4) Following cleaning at step 112 (e.g., after 10 minutes), immediately work to remove excess IPA (or other cleaning solution) from the inside of the cavity at 114. In some embodiments, because the access port (e.g., the port 46 (FIGS. 1A and 1B)) is small, step 114 may require using snippers to reveal the port. With these and related embodiments, a blunt rod or similar tool can optionally be employed to open the port, allowing for flow out of the balloon.
5) The cleaned device is allowed to dry at 116 (e.g., for a drying period on the order of 5 minutes).
6) At 118, the chamber of the printed device (e.g., the chamber 44 (FIG. 1B)) is flushed. For example, a syringe filled with fresh IPA can be employed to flush out the inside of the balloon. In some embodiments, step 118 can entail filling the printed balloon completely, cycling the syringe to induce convection. After a predetermined dwell period (e.g., 5 minutes), drain the balloon into the wash station. Repeat for each device.
7) At 120, the chamber of the printed device (e.g., the chamber 44 (FIG. 1B)) is at least partially filled and the exterior of the printed device is allowed to dry. In some embodiments, the chamber of the printed device is filled approximately ¾ of the way with distilled water. The inner lumen will expand with the water, creating uneven geometry along the inner lumen. However, this geometrical change will revert after the outside completely dries. Leave the inside filled for an extended time period (e.g., 2 hours) in a well-ventilated environment.
8) Following step 120, after 2 hours (or other drying time period), the outside of the device will be dry while the inside is full of water. The geometry will be as intended, and now the device is ready for curing at 122. In some example, curing at step 122 can include placing all devices in an appropriate cure station at a desired setting (e.g., a Form Cure station set to “Elastic 50A Resin”). The curing step 122 can extend for an appropriate length of time (e.g., 30 minutes), plus the time to heat up the curing chamber. During curing at step 122, the water will help refract the light in the chamber, allowing for an even cure. The water will also retain the geometry of the device as it cures.
9) At 124, the device(s) is removed from the cure station (e.g., Form Cure) and all water is drained. Following step 124, the device can be allowed to cool down completely and fully dry before adhering external tubing. Where desired, external tubing can be adhered to the completed balloon using a high strength adhesive glue, or by painting extra resin around the device and curing it using an external UV light.

One or more of the above steps can be modified or altered in other embodiments. For example, with some resin materials, a beneficial post-processing method 100 includes liquid immersion to allow for internal pressure in the balloon so that it does not adhere together. In other embodiments, techniques other than liquid immersion can be employed, such as pumping the balloon full of gas. In more general terms, a pressure is created inside the two walls of the balloon that keeps the walls apart (e.g., the outer and inner walls 40, 42 (FIGS. 1A and 1B)). In other embodiments, one or more of the steps of the present disclosure can be modified in accordance with properties of the balloon material. For example, a more elastic material may require more support, and thus may require another type of liquid or gas to not interrupt the cross-linking process.

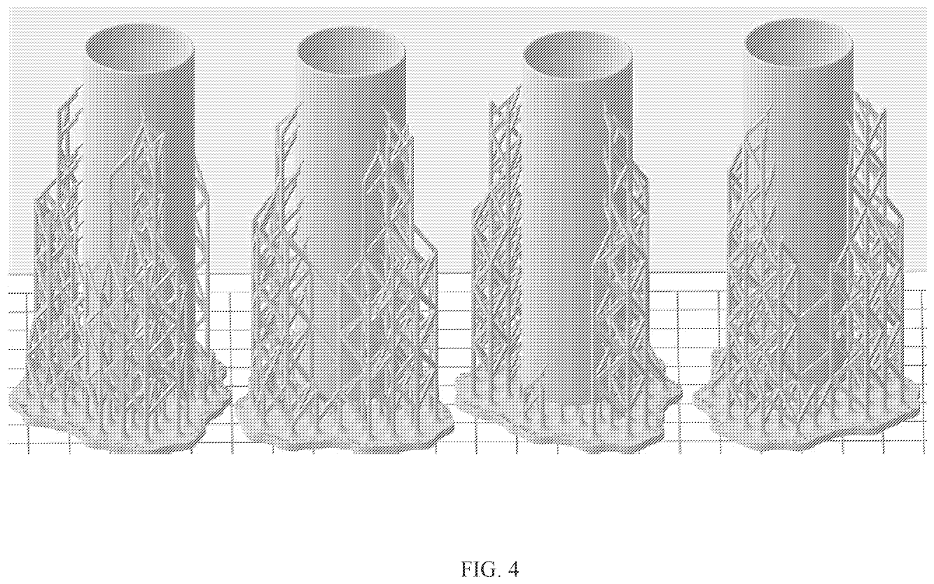

With some methods of the present disclosure, the devices can be printed on the build plate with support structures to add stability to the device while printing. It has surprisingly been found that in some embodiments, the devices printed with no inside support and with outside support will survive the post-processing steps better than the other options. With support inside the lumen, pinching would occur, causing the inside walls of the balloon to adhere to each other. Additionally, more density in support resulted in higher accounts of human error after cleaning and curing. Less support outside made the process more prone to manufacturing error, and resulted in an unstable device that could not support its own weight during curing. Shown in FIG. 4 are embodiments of optimized support design of the present disclosure, with minimal inside supports and stability from outer support arms.

Material curing also impacted the success of a finished part. As described above with respect to the method 100 of FIG. 3, in some embodiments at step 112 each device is cleaned with 99% isopropyl alcohol to remove excess uncured resin. This cleaning step 112 can include washing the inside of the device through a small hole using a syringe. After cleaning, the devices could be placed into the curing station, which runs a high intensity UV light at 60° C. for 30 minutes. However, in many designs the inner wall might stick to the outer wall during curing due to a slight warping of the material at that temperature. After this process, devices would be attached along the inside of the balloon, rendering them un-inflatable and useless. To address these possible concerns, some embodiments of the present disclosure increase success substantially by filling the balloon cavity with water (e.g., step 120). It has surprisingly been found that with this step, the manufacturing method can be more consistent.

While some examples of the present disclosure utilize 3D printing techniques to print a balloon device in an elastic resin, other manufacturing techniques can be employed. For example, the double walled balloon devices described above can alternatively be formed from a singular elastic material using molding techniques. For example, molding techniques with sacrificial material can be employed with some methods of the present disclosure. With these and related embodiments, a circumferential scaling can be employed in order to graft a sheet of some material along the inner lumen against a thicker tube of the same material. With these techniques, a chemical bond is created between two tubes of the same material, leaving an open space in between for inflation. Stated otherwise, some double walled medical balloon devices of the present disclosure can be made of two separate tubes that are sealed together at the ends. The circumferential sealing can be done chemically with a glue, heating (e.g., akin to packaging sealing), or other bonding. The sacrificial material is in reference to something being added during the molding process that is removable from the final product, such as a dissolvable support structure. Other manufacturing techniques, for example blow molding, are also available.

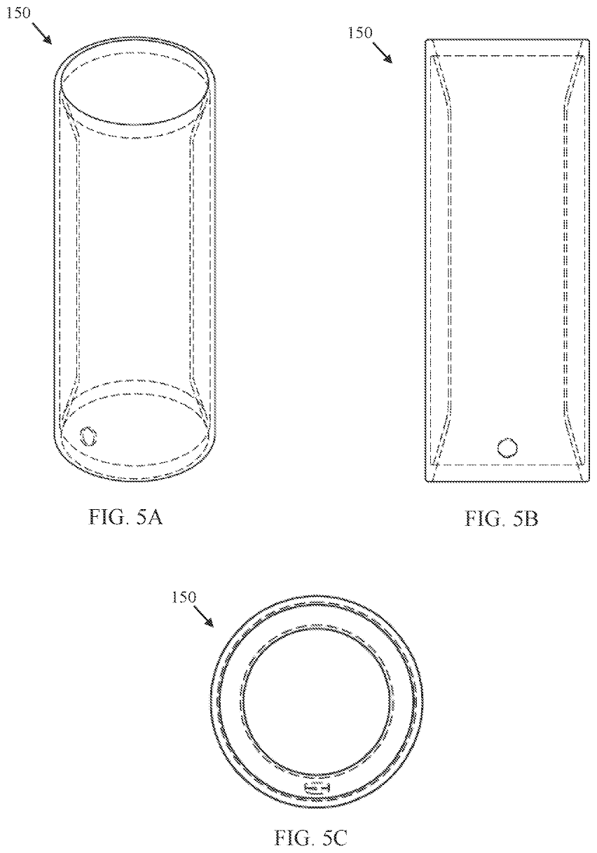

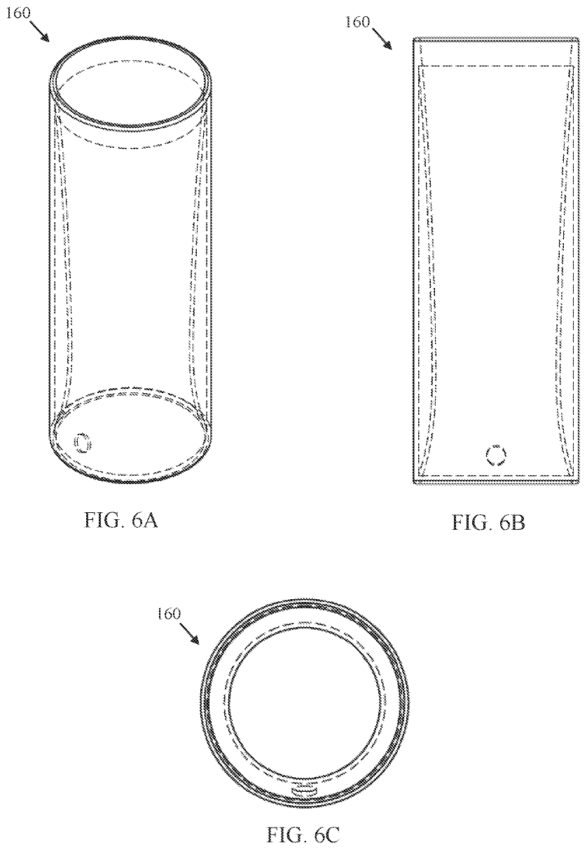

As mentioned above, a wide variety of differently-configured medical balloon devices can be made with the methods of the present disclosure, including double walled balloon devices useful with or as a singular elastic material complaint vessel implant, such as with or as any of the complaint vessel implants disclosed in Faizer et al., U.S. Pat. No. 11,883,273, the entire teachings of which are incorporated herein by reference. One example of a double walled balloon device 150 that can be manufactured using the methods of the present disclosure and useful with or as a compliant vessel implant is shown in FIGS. 5A-5C. Another example of a double walled balloon device 160 that can be manufactured using the methods of the present disclosure and useful with or as a compliant vessel implant is shown in FIGS. 6A-6C. As a point of reference, the balloon devices of the present disclosure, for example 3D printed balloon devices, can be used alone as a vessel implant or as part of a stent graft (e.g., the balloon device is disposed within a stent). With reference to the simplified representation of FIGS. 1A and 1B, with non-limiting embodiments in which the 3D printed balloon device 20 is employed as, or as part of, a vessel implant, following implant, as blood flows through the central passage or lumen 30, the conformable inner surfaces or wall 42 can deform to more closely mimic the behavior of a healthy vessel. In use, the vessel implant can be deployed on a guidewire, balloon guidewire, or other deployment system known in the art or customized for use with the vessel implant. After deployment, the guidewire can be retracted and a compressible fluid can be injected into the chamber 44. Alternatively, the compressible fluid can be injected prior to deployment. The 3D printed balloon device 20 can be deployed into the lumen of an existing stent or stent graft, or directly into a vessel. In some embodiments, the methods of the present disclosure provide a viable vessel implant homogeneously or integrally formed (printed) of a singular elastic material.

### EXAMPLES

Embodiments and advantages of features of the present disclosure are further illustrated by the following non-limiting examples. The particular materials and amounts thereof recited in these examples, as well as operating conditions and details, should not be construed to unduly limit the scope of the present disclosure.

The medical balloon or device 150 of FIGS. 5A-5C was prepared utilizing the 3D printing methods of the present disclosure using an elastic resin on a Formlabs Stereolithography (SLA) 3D printer. The balloon device had an inner wall thickness of 0.45 mm which was the smallest consistent thickness achievable with the Formlabs 3BL 3D printer. Its total length was 7 cm.

Using the post-processing procedure described above, a fully-cured device was sealed through the use of IV tubing with a diameter of 3 mm and Loctite 4013, a medical grade adhesive. A Luer-lock connector was placed in the tubing, to which a one-way valve was connected.

To prove feasibility of the device in a fully rigid flow loop, the device underwent another redesign to interface properly with the flow loop tubing. The flow loop experiments were aimed at proving reduction in blood pressure in a fully rigid system as compared to a control of the same circuit length. In this setup, experimental parameters could be examined, such as the effect of the internal balloon pressure, and device design parameters could be further analyzed, such as the survivability of implanted devices with the current geometry. A cuff was designed to allow the device to be zip-tied to tubing, allowing for the integration to be watertight.

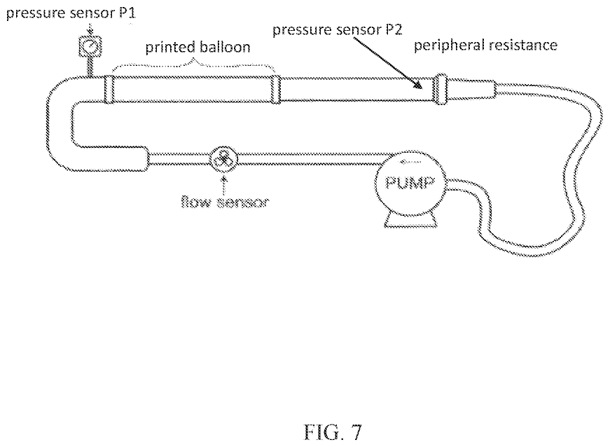

Experiments were designed to test the device versus tubing of the same length in a pulsatile flow loop as generally reflected by the schematic and photographic views of a test apparatus shown in FIG. 7 to model the response of compliant vascular implants within a vessel. The test system is akin to the test system described in Faizer et al., U.S. Pat. No. 11,883,273, the entire teachings of which are incorporated herein by reference, with the addition of a second downstream pressure sensor (labeled as “P2”) and can be used to model the pressure versus time for various diameters and thicknesses (indicative of compliancy) of tubes. A pulsatile flow rate can be imposed by the pump. The first two experiments yielded no results due to leaking. In a third experiment, the device inner wall yielded to the stress of the flow, breaking inside the flow loop due to pinching of the interface. With several redesigns focusing on reducing the stress at the interface site, the 7 cm device was tested on the flow loop without breaking or leaking, and hemodynamic data was collected. The next experiment utilized more devices to characterize the impact of changing the internal pressure of the device. By putting in volumes of air equal to 2 mL, 3 mL, 5 mL, 7 mL, and 8 mL, the change of dynamics was observed as the device was filled up more.

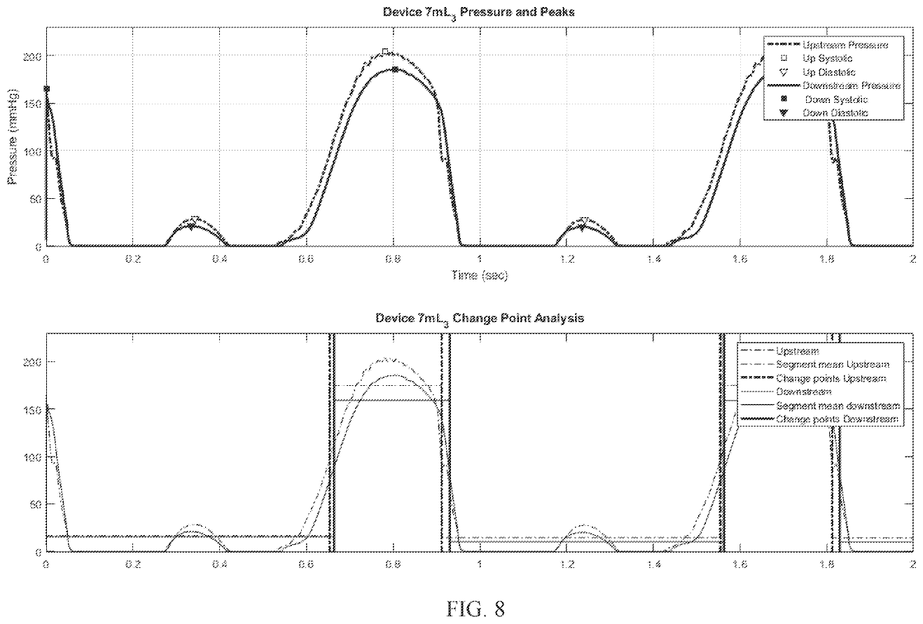

Peak analysis was performed to determine the systolic and diastolic pressures of the control and the devices. Each data point in the analysis was shown to be statistically significant with a paired t-test with a p-value ranging from 10-4 to 10″ 138. The raw data with each of the identified peaks and change points is shown in FIG. 8 (that otherwise provides peak and change point analysis on smoothed data). Change point analysis was used to identify the points of highest pressure change for pulse wave velocity (PWV) calculations.

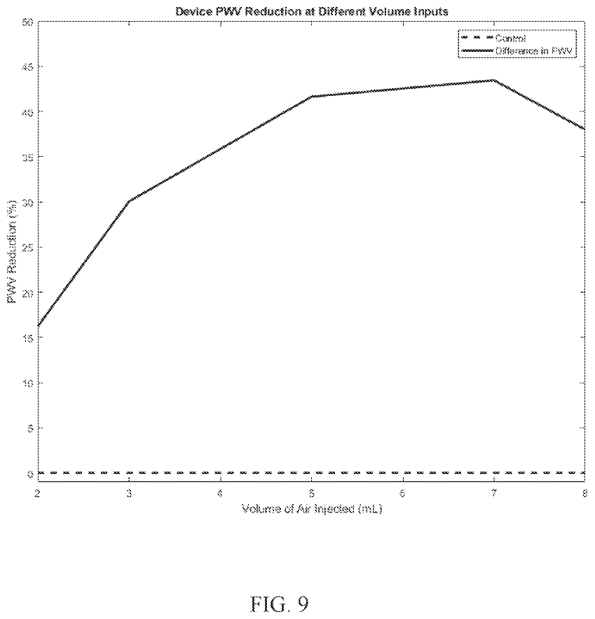

The peaks were used to calculate the reduction in systolic pressure for each sample. With higher internal pressure, the device showed a higher systolic pressure reduction, although at 8 ml volume injected, the systolic pressure changed to be closer to that of the non-deformable control. It was determined that the device would have diminishing returns at higher pressures due to the increase of stiffness of the device. At a volume of 7 mL, the device caused a systolic pressure reduction of an average of 17.5 mmHg, which was just under the target of 20 mmHg. Importantly, this measurement was recorded proximal to the device, demonstrating that the effect was not due to resistance to flow through the device, but instead due to successful capacitance function (absorption and redistribution of the pressure wave). Device upstream and downstream pressure reduction compared to control is shown in FIG. 9.

The change points were then used to quantify the change in pulse-wave velocity (PWV) compared to the control over each of the samples. This was considered an important parameter as PWV is a recognized and validated approach to assess aortic stiffness. These change points marked t1 and t2 as the upstream and downstream reference points for time for each waveform. The velocity was found by calculating the change in distance over the change in time using the length of the device (d). The equation below was used to calculate the PWV as:

$$PWV ( {ms}^{- 1} ) = \frac{d}{t 2 - t 1}$$

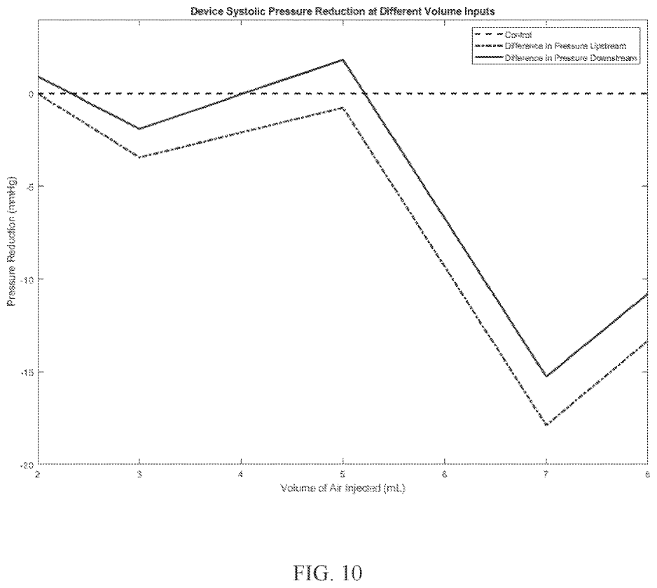

The PWV reduction was up to over 40% from the control as seen in FIG. 10. This was past a target of 10-15% reduction and shows promise for using the device to control this parameter in vivo.

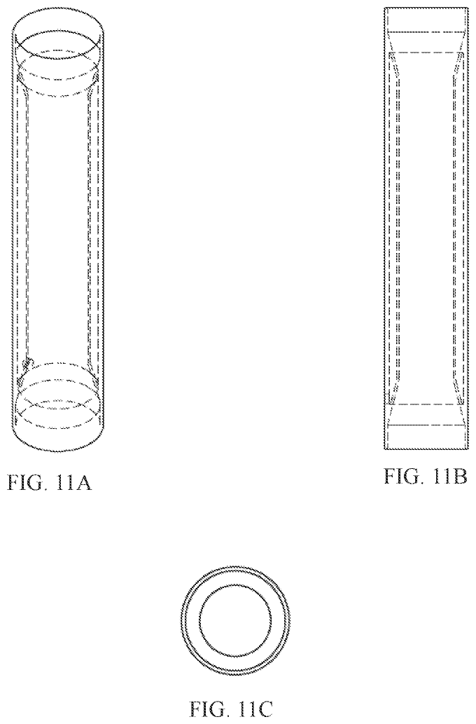

As the manufacturing methods had been explored thoroughly, a 14 cm device was next prepared using the 3D printing methodologies of the present disclosure. A device was designed as shown in FIGS. 11A-11C and printed using the framework set by the shorter devices. After tweaking the support structure parameters, several devices were manufactured with no inner wall adhesion and with the expected functionality. Instead of using an adhesive to affix the external tubing to the device, uncured resin was applied to the interface and the tubing was inserted prior to the post-curing step, allowing the tubing to be fully integrated into the device. The benefits of this method were that the material was homogenous and the tubing was fully integrated with a material that would expand and contract with the device.

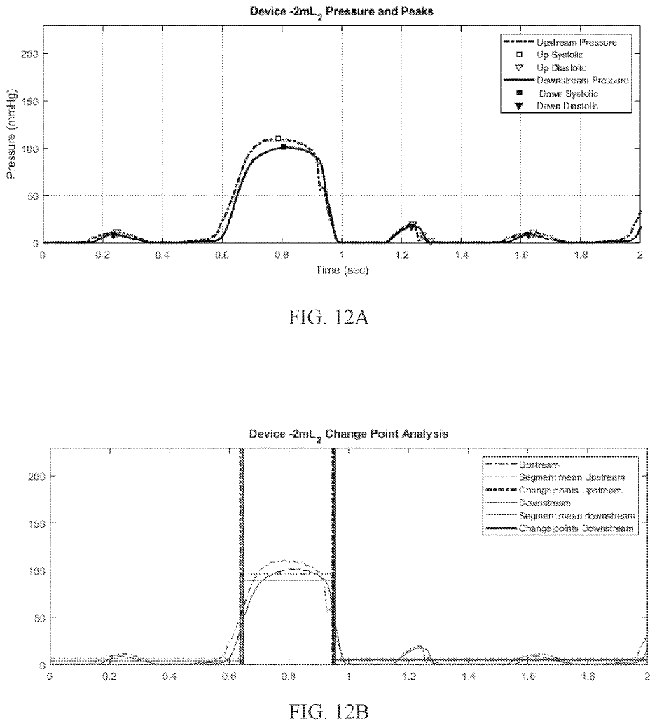

Further testing was conducted using this full-length, 14 cm ballooning device. This device was tested with internal vacuum in addition to internal pressure. FIGS. 12A and 12B depict data analysis which was conducted for each sample. Peak pressures (FIG. 12A) and change point (FIG. 12B) analysis were analyzed to depict flow dynamics on a smoothed set of data.

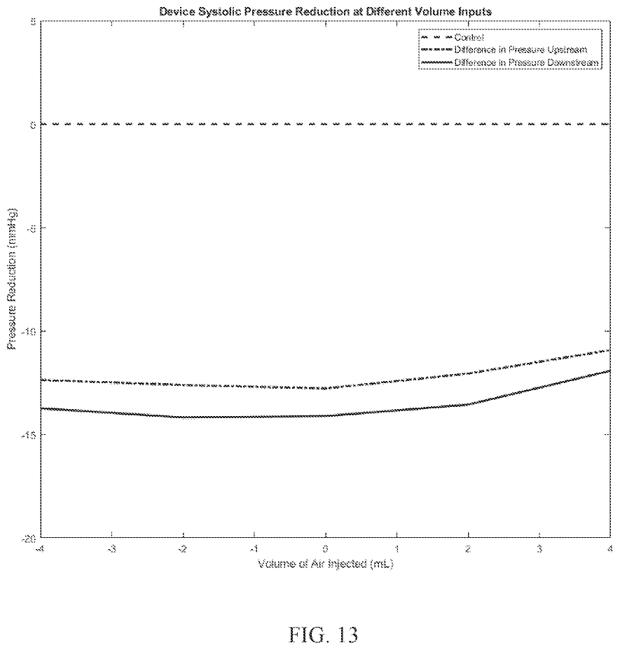

In the peak analysis shown in FIG. 13, the systolic pressure reduction was within the same range for each of the volume inputs. Since the systolic pressure upstream difference was less than the downstream difference, it could not be definitively concluded that obstruction did not affect this pressure dynamic. Similarly, since the effect of the leaking is unknown, it could have also contributed to a dramatic loss in pressure over the length of the device.

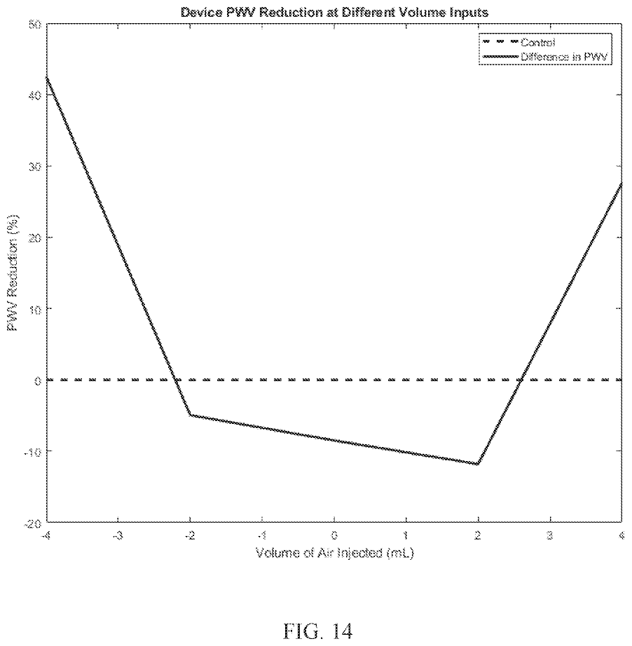

In the PWV analysis of FIG. 14, the changing volume of air depicted high PWV changes at higher vacuum and higher pressure. This could be a useful control for the device.

As another example, the medical balloon or device 160 of FIGS. 6A-6C was prepared utilizing the 3D printing methods of the present disclosure. The device 160 has a teardrop-type profile to customize pulse wave velocity and the systolic blood pressure reduction as described above. The device 160 is a design based on fluid dynamic simulations which allow for small changes in flow with large changes in pressure. Changing this geometry paves the way for patient specific vascular devices, a novel concept that could be cost effective using 3D printing technology.

The systems and methods used to construct the devices of the present disclosure are novel, with the optional 3D printing techniques providing one solution to a geometrical problem. The benefit of 3D printing is of particular interest as it allows for customizability in a way achievable only by using this manufacturing method. The geometry could fit perfectly within a patient's blood vessels due to the flexibility in design. The patient could receive a device that reduces blood pressure to a predefined extent, adding more precision to the treatment of cardiovascular disease. Importantly, 3D printing could allow for development of specific morphologies of the inner balloon to minimize drag and turbulence.

Some non-limiting aspects of the present disclosure accomplish two specific aims for a singular elastic material double walled balloon to be employed as a vessel implant, for example as an implantable, inflatable device that sits in the descending aorta of a patient. A first aim is to reduce systolic blood pressure and pulse wave velocity compared to a control of a stiff aorta. The short device managed to reduce blood pressure statistically significantly by over 15 mmHg in a rigid flow loop compared to a completely stiff circuit of the same length. The PWV was reduced by over 40% as compared to the control circuit. While testing did not include a diseased elderly aorta of high stiffness for comparison, these results prove that the device can reduce pressure and PWV significantly without obstructing flow.

A second aim was to manufacture the device in such a way that it could be compressed and delivered in an endograft delivery system. The devices of the Examples section compress down into an area smaller than current delivery sheaths, meaning that this is possible.

The systems and methods of the present disclosure provide a marked improvement over previous designs. Medical balloon devices were printed using a stereolithography 3D printer in an elastic resin in some embodiments. The so-prepared device was interfaced with a syringe and shown to inflate radially inwards and deflate radially outwards without any pressure loss. The present disclosure provides a framework for using an elastic and biocompatible material to prepare or manufacture, for example, a singular elastic material endoprosthesis device to be implanted in the human body. The optional 3D printing systems and methods of the present disclosure provide a solution to geometry issues posed by manufacturing a hollow elastic cylinder, which could be used, for example, in venous or aortic stent-graft devices. The systems and methods of the present disclosure further allow for the creation of patient-specific devices tuned for different vasculature diameter and length. Additionally, by controlling the thickness of the device and changing its lumen profile, the reduction in pressure could be regulated by the geometry such that a patient can receive a device that reduces their blood pressure by a specific and predetermined amount. Vascular dynamics may change with various device parameters such as positive or negative internal pressure, device length, and device wall thickness which adds another level of customizability. The methods of the present disclosure facilitate customizability to the resultant double walled medical device balloon; as explained above, with different custom profiles and inflation parameters, the systolic pressure and PWV could be controlled. The systems and methods of the present disclosure can be beneficial for other end-use applications, such as for venous or other external applications and could be used externally in areas such as urology.

Although the present disclosure has been described with reference to preferred embodiments, workers skilled in the art will recognize that changes can be made in form and detail without departing from the spirit and scope of the present disclosure.

1. A method for manufacturing a medical balloon device, the method comprising:
preparing the medical balloon device from a singular elastic material.

2. The method of claim 1, wherein the medical balloon device has a double wall construction.

3. The method of claim 2, wherein the medical balloon device has a hollow, cylinder shape defining a central passage, and further wherein the double wall construction includes an outer wall and an inner wall combining to form a chamber.

4. The method of claim 3, wherein the medical balloon device further includes a port through the outer wall and in fluid communication with the chamber.

5. The method of claim 4, wherein the step of preparing includes 3D printing the medical balloon device.

6. The method of claim 5, wherein the step of 3D printing includes:
orienting a modeling of the device to be printed relative to a build plate of a 3D printer such that a cylindrical edge of the model is perpendicular to the build plate.

7. The method of claim 5, wherein the step of 3D printing includes:
orienting a modeling of the device to be printed relative to a build plate of a 3D printer such that a port of the model is proximate the build plate.

8. The method of claim 5, wherein the step of 3D printing includes:
orienting a modeling of the device to be printed relative to a build plate of a 3D printer; and
adding support structures to an exterior wall of the model.

9. The method of claim 5, wherein the step of 3D printing includes:
printing a modeling of the device to a build plate of a 3D printer;
removing the printed device from the build plate;
cleaning the printed device;
at least partially filling the chamber of the printed device with water; and
allowing an exterior of the printed device to dry while the chamber remains at least partially filled with water.

10. The method of claim 5, wherein the step of 3D printing includes:
printing a modeling of the device to a build plate of a 3D printer;
removing the printed device from the build plate;
cleaning the printed device;
creating a pressure within the chamber of the printed device with water; and
curing the printed device while the chamber remains pressurized.

11. The method of claim 10, wherein the step of creating pressure includes at least partially filling the chamber with water.

12. The method of claim 11, wherein the step of curing includes the chamber remaining at least partially filled with water.

13. The method of claim 1, wherein the medical balloon device is a compliant vessel implant.

14. The method of claim 13, wherein the step of preparing includes customizing a profile and inflation parameters of the compliant vessel implant.

15. The method of claim 13, wherein the compliant vessel implant is formed as a homogeneous body of a singular material.

16. The method of claim 1, wherein the medical balloon device is an aortic endoprosthesis.

17. The method of claim 1, wherein the step of preparing includes printing the medical balloon device by a stereolithography printer.

18. The method of claim 1, wherein the singular elastic material is a biocompatible resin.

19. The method of claim 1, wherein the step of preparing includes customizing a profile and inflation.
